(19) United States
(12) Patent Application Publication
(10) Pub. No.: US 2009/0255543 A1
(43) Pub. Date: Oct. 15, 2009

(54) Method of chiropractic treatment

(76) Inventor: Masumi Oyama, Fukuyama-city (JP)

Correspondence Address: MEREK, BLACKMON & VOORHEES, LLC, 673 S. WASHINGTON ST, ALEXANDRIA, VA, 22314, US

(21) Appl. No.: 12/081,252

(22) Filed: Apr. 14, 2008

(51) Int. Cl. A61B 19/00

(52) U.S. Cl. 128/898

(57) ABSTRACT

A method of chiropractic treatment in which a patient's head and legs are moved along predetermined paths each having plural transit points on a circle having its center along the axis of the patient's spine. Each transit point has a counterpart transit point located at a position symmetric with the transit point with respect to the center of the circle. Each path has plural sub-paths each extending along a curve from a transit point and through the center of the circle to a counterpart transit point, wherein, during the movement of the patient's legs along a sub-path, the head is moved along a different sub-path starting from a transit point corresponding to the counterpart transit point of the movement of the legs.

### BACKGROUND OF THE INVENTION

a) Field of the Invention

This invention relates to a method of chiropractic treatment. More particularly, the invention relates to a treatment whereby spinal alignment is stabilized in a non-traumatic way, indirectly by moving a person's head and legs, thereby arranging the spine so that the daily pains accumulated by movement or stress can be reduced or eliminated.

b) Brief Description of the Prior Art

People always have a tendency to use the same side of the body when doing physical exercise or physical work. This produces an imbalance in the spine and affects sciatic nerve pressure and the like.

### OBJECTS AND SUMMARY OF THE INVENTION

It is therefore an object of the present invention to provide a chiropractic treatment to reduce nerve pain caused by imbalance of the spine.

Chiropractic treatment according to the present invention comprises

a) holding a patient's head and legs;

b) moving the patient's head and legs simultaneously each along a path, wherein each path has plural transit points on a circle the center of which is located at the axis of the patient's spine, and each transit point has a counterpart located at a symmetrical position with respect to the center of the circle, wherein the path has plural sub-paths each extending along a curve from one transit point and through the center of the circle to another transit point, wherein, during the movement along a first sub-path at the legs, the movement at the head is along a different sub-path that starts at a transit point corresponding to the transit point where the first sub-path along which the legs are moved terminates, wherein each path is in a plane perpendicular to the axis of the spine of the patient, and wherein, among the transit points, two are located at opposed horizontal positions and two are located at opposed vertical positions;

c) synchronizing the movement so that the head and legs pass through the center of the circle and reach a terminal transit point at the same time; and

d) continuously moving the head and legs along succeeding sub-paths, wherein each succeeding sub-path starts at the transit point that is the terminus of the previous sub-path.

### BRIEF DESCRIPTION OF THE DRAWINGS

The invention will now be illustrated by means of the annexed drawings, it being understood that they are given only for the purpose of illustration and not for restricting the scope of protection, which is defined by the appended claims.

FIG. 1 is a schematic side view of a treatment according to the invention;

FIG. 2 shows an example of a path along which the legs and head are moved;

FIGS. 3A and 3B show further examples of the path;

FIG. 4 is a schematic view showing the effects of the treatment on the human body;

FIG. 5 is a side view of an apparatus for swinging the head of a patient and the feet;

FIGS. 6A-6D show further examples of the path;

FIG. 7 is a cross sectional view of FIG. 5.

FIG. 8 is a front view of a pinion, gear and pin of the apparatus of FIG. 5;

FIG. 9 comprises views showing the movement of the apparatus of FIG. 5;

FIG. 10 is a locus diagram of the movement of the apparatus of FIG. 5; and

FIG. 11 is a side view of an apparatus for use with the method of the present invention, the apparatus including two of the apparatuses of FIG. 5.

### DETAILED DESCRIPTION OF THE PREFERRED EMBODIMENT

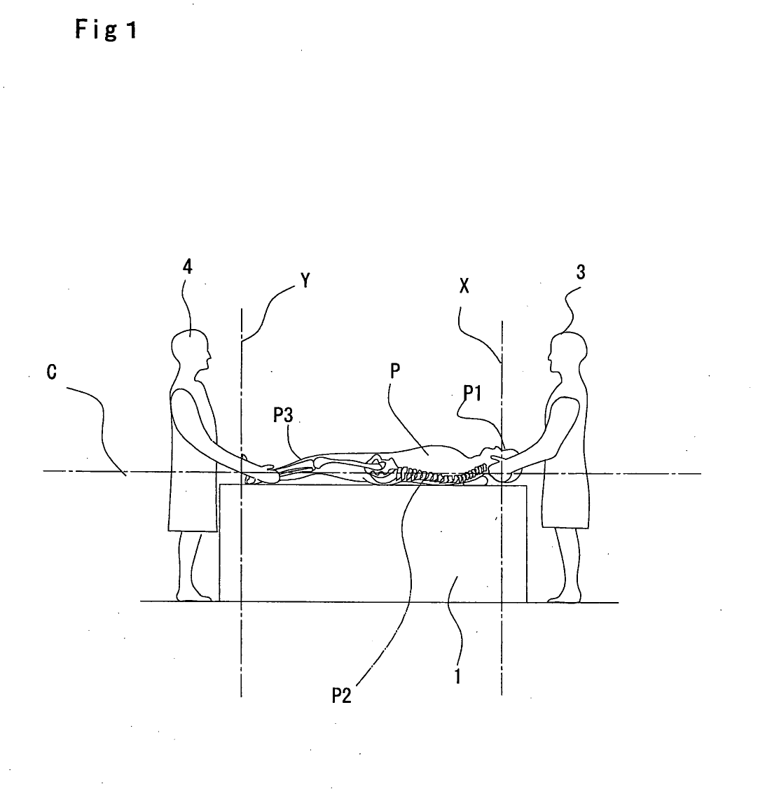

The preferred embodiment is now described by referring to the figures. In FIG. 1, the patient P lies on his back on the bed 1. According to the embodiment to be described, because the patient's head P1 will be moved right, left, upward and downward, it is preferable to have enough space for that movement. To achieve this, the shoulder of the patient is lifted slightly by putting a pillow 2 under the shoulder. In the same way, another pillow is put beneath the buttocks of the patient. Those pillows have a rectangular shape or a column shape. A therapist 3 stands at the patient's head and another therapist 4 at the patient's legs. The bed 1 has open space on its sides so that the two therapists can reach the patient's body from any direction. For this purpose, a conventional treating bed can be used by using two pillows.

One therapist 3 holds the patient's head with both hands. The other therapist 4 holds the patient's heels. The two therapists move their hands along a predetermined path.

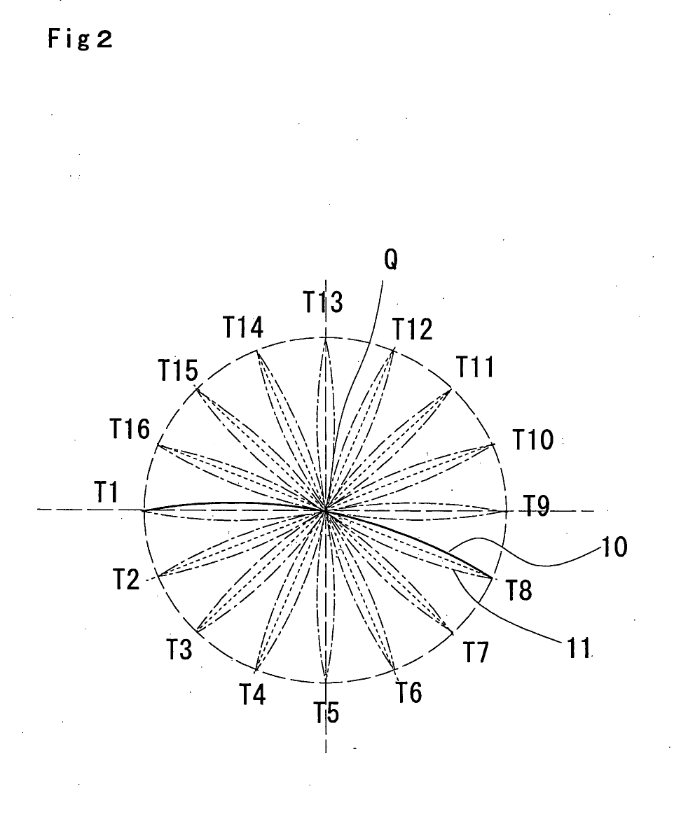

FIG. 2 shows the predetermined path along which therapists move their hands. The path has an even number of transit points T1, T2, . . . T16 lying on a circle R having a center at a point Q. Those points are positioned every 22.5 degrees along the circle. Among them, four points—T1, T5, T9 and T13—are located at horizontal positions and vertical positions. The path consists of plural sub-paths 10, in this example, sixteen sub-paths. One sub-path starts at one of the transit points and ends at another one of the transit points, a transit point that is located at 157.5 degrees around the circle from the starting transit point. Assuming that the starting point is T1, then the sub-path terminates at T8. After traveling all sixteen sub-paths, the movement returns to the starting point T1. In each sub-path, the shape of the route is a curve, or an arc, and passes through the center Q of the circle.

Succeeding sub-paths start from the transit point where the previous sub-path terminated. Assuming that the previous sub-path terminated at T8, then point T8 becomes the starting point for the succeeding sub-path. In the succeeding sub-path, the route also has a curved shape and passes through the center Q of the circle before reaching another transit point that will become next the starting point. In like manner, the sub-paths continue and return to the starting point where the first sub-path initiated.

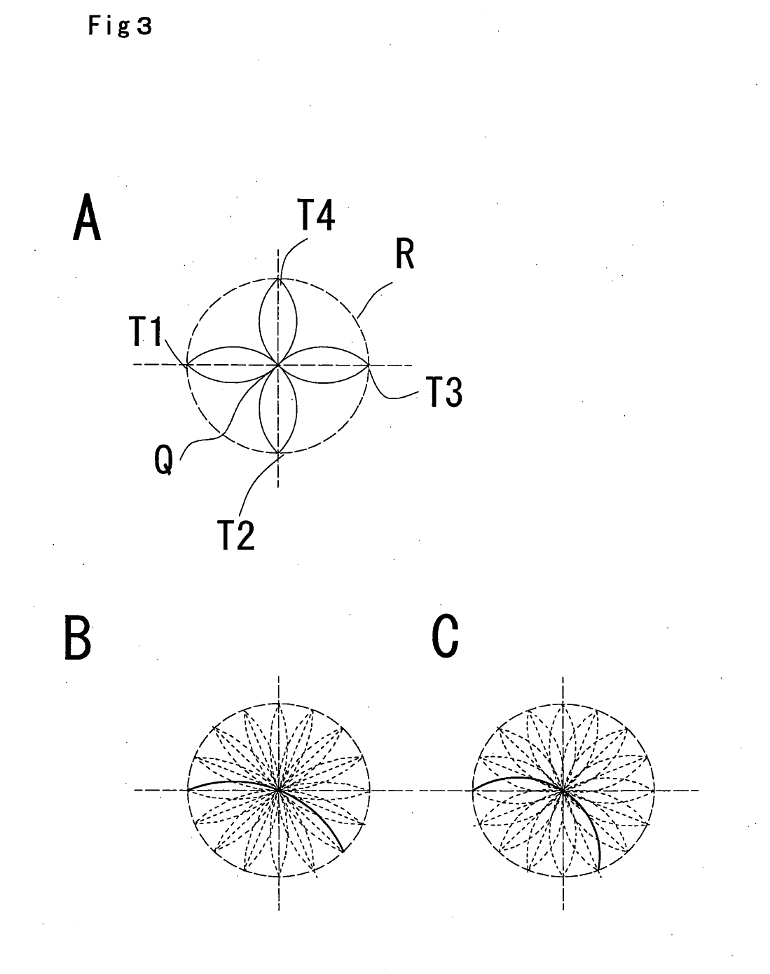

FIG. 3A shows another type of path where the path has four transit points. In this path, the transit points are arranged every 90 degrees, and four sub-paths connect the transit points next to each other. FIGS. 3B and 3C show other types of paths that also can be used with the present embodiment. However, it is common to the paths in FIGS. 3A-3C that four transit points are located at horizontal positions and vertical positions on the circle R.

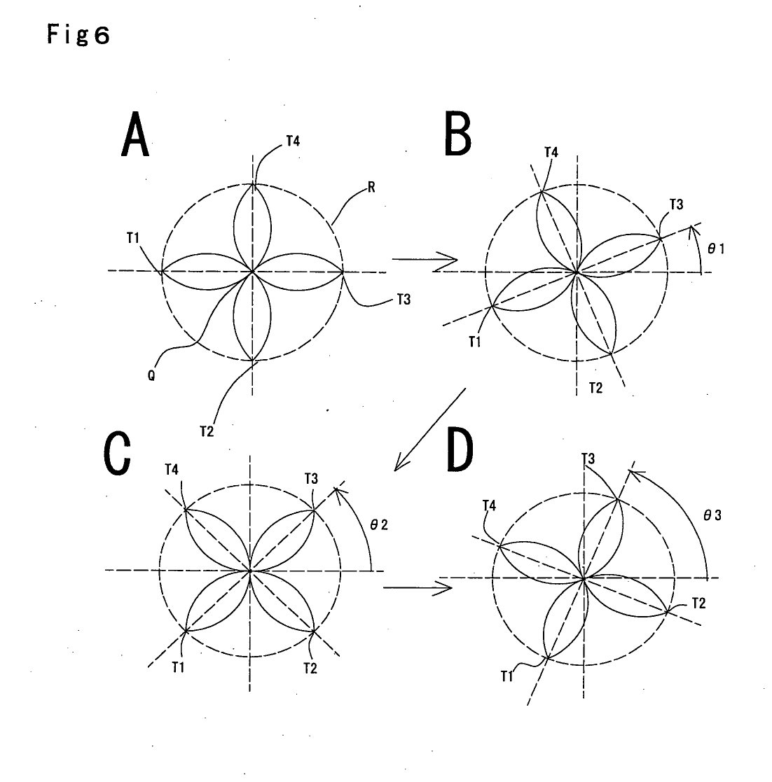

FIGS. 6A-6D show another type of path. This path utilizes the path showed in FIG. 3A. The difference is that the path in FIGS. 6A-6D comprises a plurality of the paths of FIG. 3A, each of which turns through a certain angle, such as θ 1, θ 2 or θ 3. In FIG. 6B, θ 1 equals 22.5°, in FIG. 6C, θ 2 equals 45° and, in FIG. 6D. θ 3 equals 67.5°. These four sets of the path form a single path. Alternatively, the rotational angles θ 1, θ 2 and θ 3 can be selected in a clockwise direction.

Now, according to the treatment method, the patient's head and neck are placed on the axis of the spine. The legs, however, are lifted slightly. It is not necessary that the legs be aligned with the axis of the spinal, because the legs are far from the spine and many joints exist therebetween.

It is deemed that the positions of the head and legs on the axis C (FIG. 1) of the spine are an initial position before the treatment starts and considered that the position corresponds to the center Q of the circle. The planes X and Y (FIG. 1) are defined at the position of the head and legs. The planes X and Y are perpendicular to the axis C (FIG. 1) of the patient's spine. In those planes, the therapists move the patient's head and legs along their assigned paths.

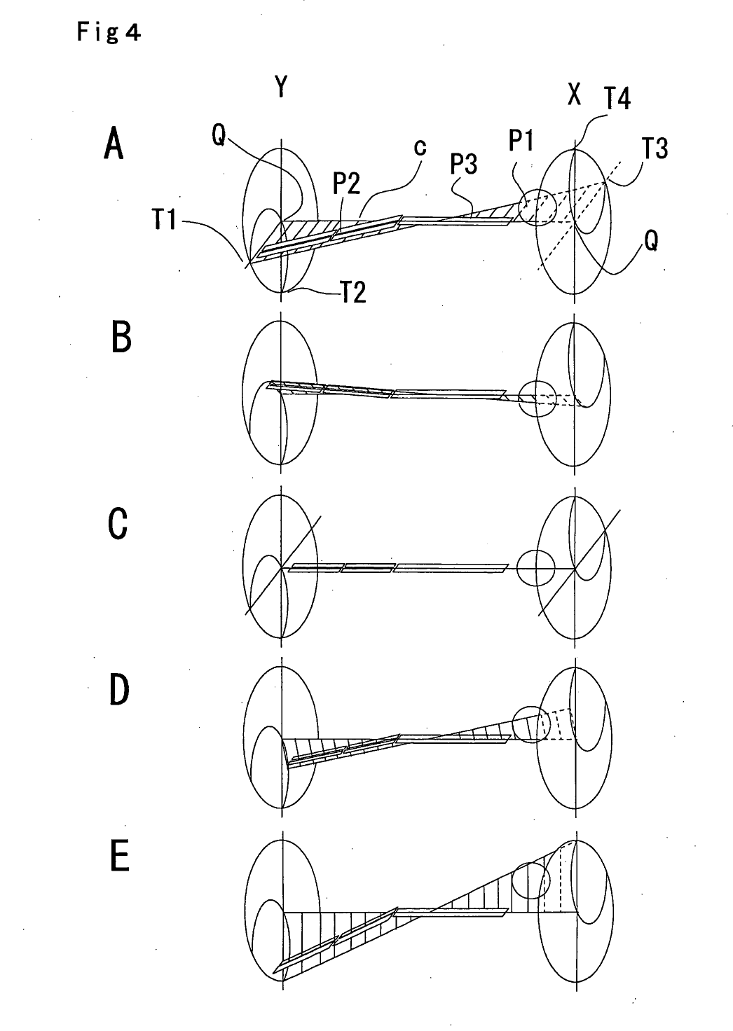

If the therapists select the path depicted in FIGS. 3A-3C, and if one therapist selects the starting point T1 from which to start the treatment, then the other therapist selects point T3 as the starting point. The two points are located symmetrically on the circles. The two therapists move the head and legs from the center position Q to the transit points T3 and T1 respectively (FIG. 4A) to set an initial position of the treatment, and then start to move the head and legs along the curved sub-paths simultaneously from those transit points. FIG. 4 shows the movement of the patient's head P1, spine P3 and legs P2. The speed of the movement is about one second for traveling through a sub-path. It is important that the therapists' movements are symmetrical with one another and pass through the center Q at the same time. It is also important that their movements are synchronized to each other. When the body of the patient is too heavy to carry, both therapists are allowed to exceptionally start from T2 and move to T1 or T3 thereafter. The radius of the circles on the plane X is different from that of the circles on the plane Y, since the head is more closely connected to the spine. For this reason, the circle on the plane X is smaller than that on the plane Y. If the patient feels pain when the therapist sets the head at certain starting point, the therapist has to make the circle on the plane X smaller. The positions of the head and the legs move toward or away from the center Q, but these positions are always symmetrically located while traveling the sub-path. At the end of the sub-path, the directions of the head P1 and and the legs P3 rotate 90 degrees.

The therapists can decide to change the size of the circle R, when the therapists find that changing the size of the circle R, for example, to a smaller one will be more appropriate than keeping the size of the circle R. If this decision is made, the succeeding sub-path starts from a revised transit point that is shifted toward the center Q.

After the next sub-path done, the directions in which the head P1 and the legs P3 travel rotates a further 90 degrees. When travel along all of the sub-paths, that is, along the entire path is completed, the directions of the head P1 and the legs P3 has rotated 360 degrees.

The movements the two therapists make do not directly affect the spine of the patient, but rather the movement is indirectly transmitted through pelvic and neck joints in the manner of a propagating wave. In all movement of the head and legs toward or away from the spine, the wave caused by the movement extends to the spine and cures imbalance of the spine. Because the sub-paths start from four or more directions and trace curves, any direction of imbalance occurring in the spine can be treated.

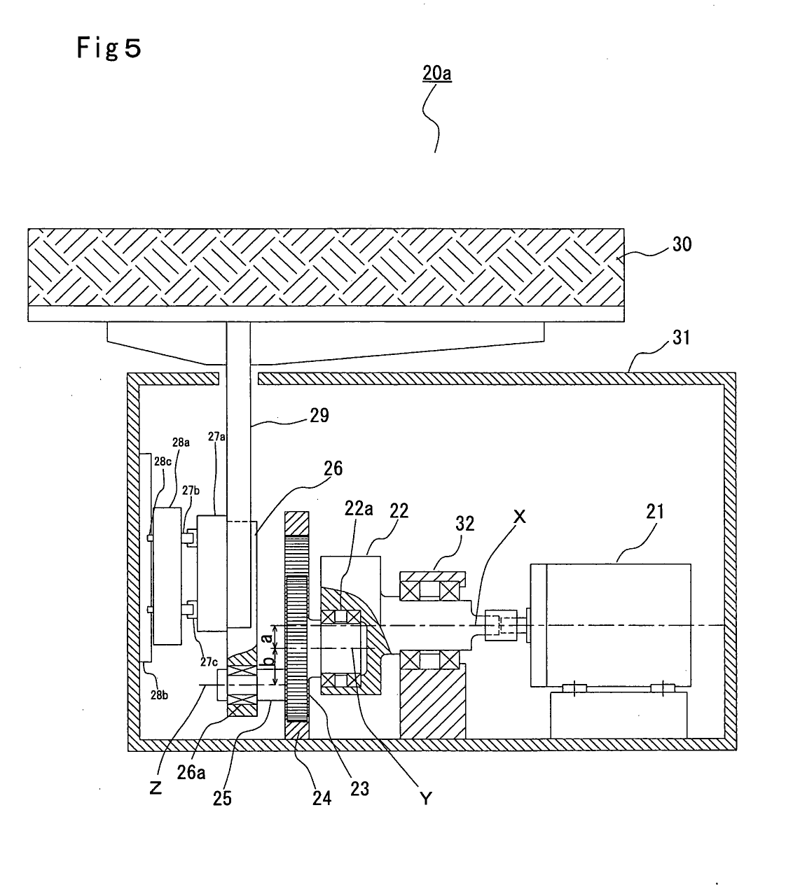

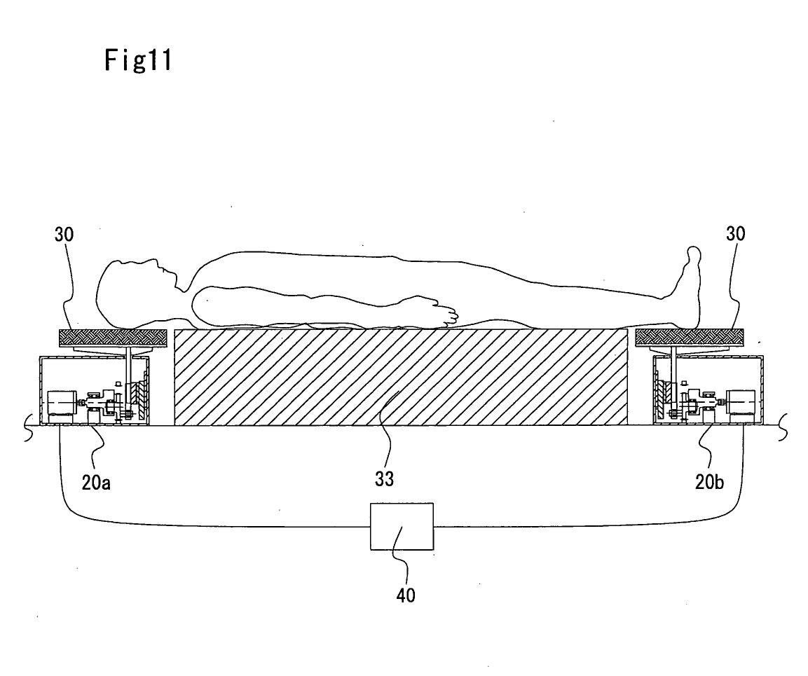

Another embodiment of the invention will be described with reference to the drawings. FIGS. 5 and 11 show an embodiment employing an apparatus.

As can be seen in FIG. 11, the apparatus comprises two swing devices 20a and 20b. These are installed near opposite ends of a bed 33. Each has a swing table 30 so that the patient can put his head or feet thereon. A controller 40 controls the swing devices 20a and 20b.

FIG. 6 shows a transverse view of the swing device 20a. The swing device 20b has the same structure as the swing device 20a, which will be described. In a casing 31, a servomotor 21 and a bearing case 32 are provided. A rotating shaft 22 is supported by the bearing case 32 and connected to the servomotor 32 to rotate around the rotation axis X. At the front end of the rotating shaft 22, a flange is formed, and a pinion 23 is rotatively mounted on the flange through the bearing 22a. The rotation axis Y of the pinion is shifted by an amount “a” from the rotation axis X of the shaft 22. A hollow gear 24 having teeth on its inside is installed concentrically with the rotating shaft 22, and the pinion 23 engages the teeth of the gear 24. The ratio of the number of teeth of the gear 24 to that of the pinion 23 is 4 to 3. A pin 25 is mounted on the front face of the pinion 23 at a position Z that is eccentric from the rotation axis Y by an amount “b”. A connecting bar 26 is connected to the pin 25 through a bearing 26a. A movable table 27a is fixed to the connecting bar 26 for guiding lateral movement together with legs 29 of a swinging table 30. The movable table 27a has guide blocks 27c that move in a lateral direction on a pair of slide rails 27b. The slide rails 27b are mounted on a movable table 28a having guide blocks 28c. The guide blocks 28c move along a slide rail 28b aligned in a vertical direction and fixed on the casing 31 for guiding vertical movement of the movable table 28a.

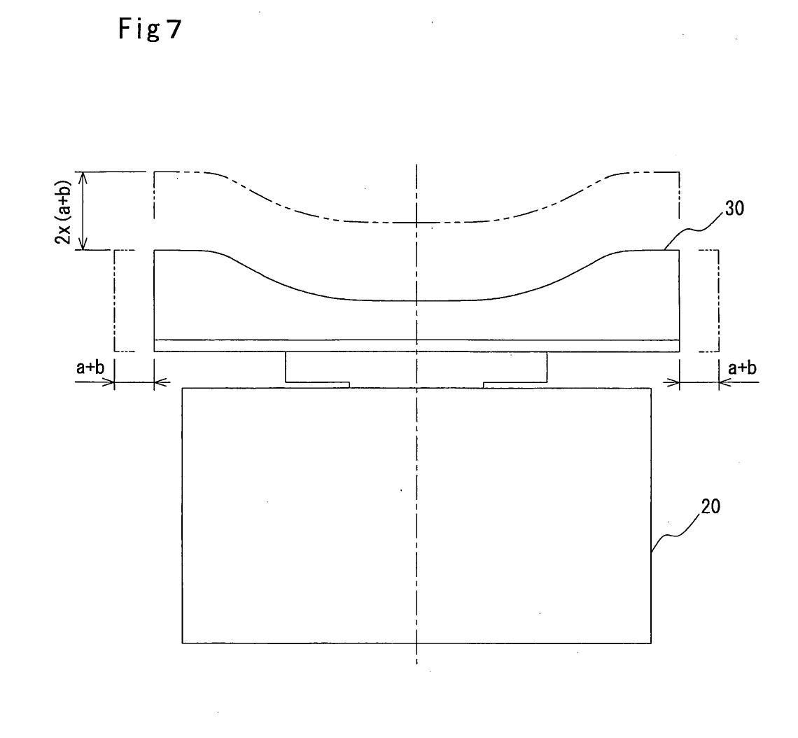

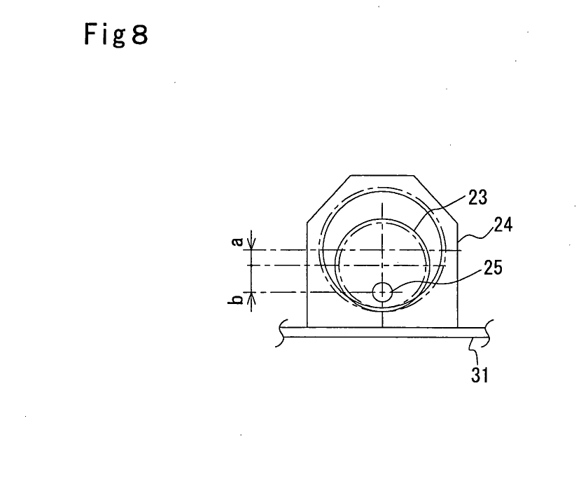

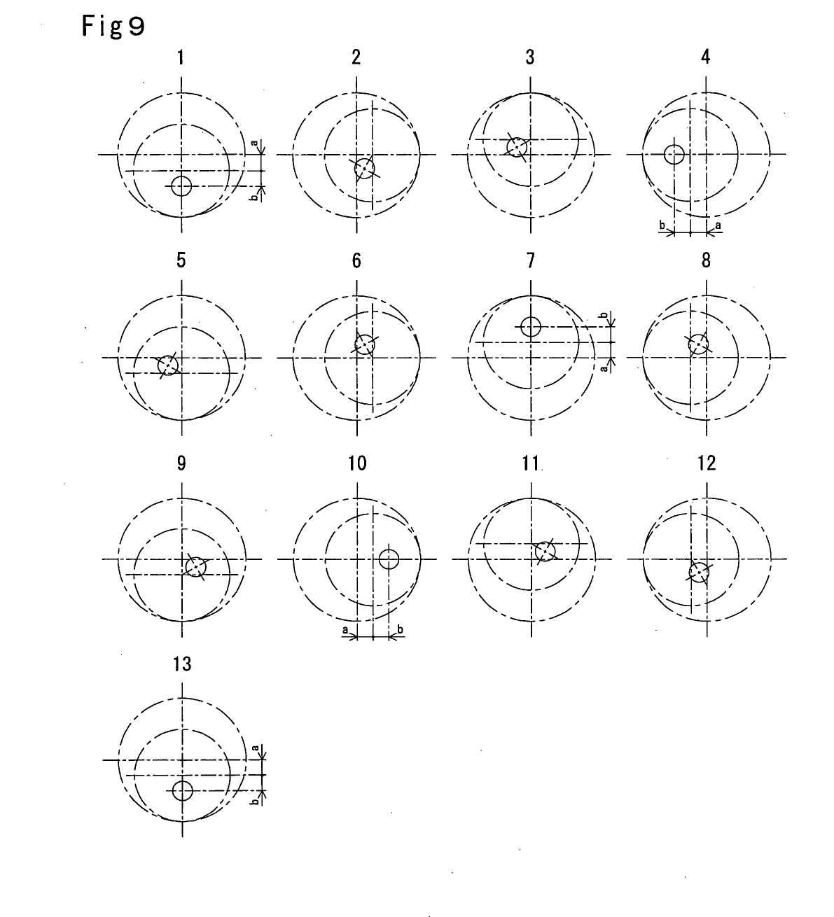

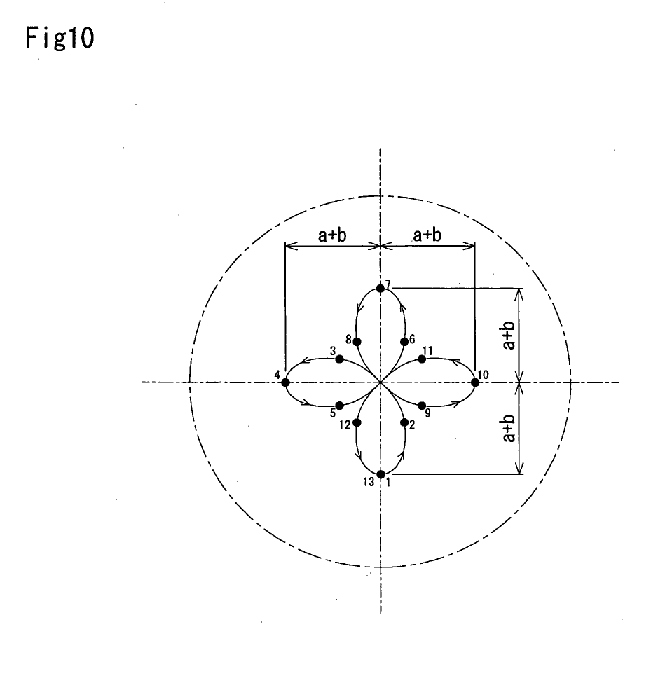

Next, the operation of the apparatus will be explained. The controller 40 initializes the swing devices 20a and 20b. The pin 25 of the swing device 20a locates at one of the four horizontal positions or vertical positions relative to the rotation axis Y of the pinion 23. If the pin 25 of the swing device 20a locates at one of the horizontal positions, then the pin 25 of the swing device 20b locates at one of the vertical positions. This control can be done by pulsing the servo-motors 21 of the two swing devices. FIGS. 5, 7 and 8 show the state in which the pin 25 locates at the lower vertical position. FIGS. 9 and 10 show a movement of the pin 25 while the pinion 23 travels inside the gear 24 counterclockwise, when viewed from the shaft of the servomotor 21, and returns to the initial lower vertical position. When the servomotor 21 rotates counterclockwise, the shaft 22 rotates counterclockwise. The pinion 23 rotates clockwise around the axis Y and rotates around the axis X counterclockwise inside the gear 24. FIG. 9 shows the positions of the pin 25 where the pinion 23 rotates inside the gear 24 every 90 degrees. FIG. 10 shows a movement of a cycle of the pin 25. The numbered positions 1 to 13 in FIG. 10 correspond to the numbered positions 1 to 13 of the pin 25 in FIG. 9.

The pin 25 moves from the lower position to the left along the center of the rotating shaft 22 or the neighborhood, then moves to the top along the center or the neighborhood, thereafter passes the right, and returns to the initial start position. The pin 25 moves by “a”+“b” to the left, 2×(“a”+“b”) to the top and “a”+“b” to the right when the lower position is the start position. Alternatively, the initial position can be the top position, the right position or the left position, and the rotation direction can be reversed from that of FIG. 9 or FIG. 10. The movement of the pin 25 is changed to lateral movement and vertical movement by the table 27a and the table 28a. The swing table 30 follows the path that the pin 25 travels.

Comparing FIG. 3A and FIG. 10, the paths are not identical but similar. It is understood from these that the path not only comprises an arc, but also comprises a curve that is continuous and passes through the center position. However, it is not necessary that a sub-path and a succeeding sub-path connect with a continuous curve at a transit point.

The swing device just described draws the path by mechanical rotation, but the shaft 22 and the pinion 23 can be substituted by a servo actuator to move the movable tables 27a and 28a in lateral and vertical directions independently. In such an implementation, the controller 40 can control the swing devices 20a and 20b to trace the path showed in FIG. 3A.

What is claimed is:

1. A method of chiropractic treatment of a person lying on the person's back, comprising:
a) holding the head and the legs of the person;
b) moving the head and the legs simultaneously, each along a path having plural transit points lying on a circle, the center of which is located at the axis of the person's spine, each transit point having a counterpart transit point located at a position symmetric with the transit point with respect to the center of the circle, wherein the path has plural sub-paths each of which extends along a curve from a transit point and through the center of the circle to another transit point, wherein, while the legs are moved along a sub-path, the head is moved along a sub-path different from the sub-path along which the legs are moved, the sub-path along which the head is moved starting at a transit point corresponding to the counterpart transit point of the sub-path along which the legs are moved, wherein each path is in a plane that is perpendicular to the axis of the spine of the person, wherein among the transit points of each path are two transit points that are in horizontal alignment with one another and two transit points that are in vertical alignment with one another;
c) synchronizing the movement so that the head and legs pass through the center of their circle at the same time and reach a counterpart transit point at the same time; and
d) continuously moving the head and legs along succeeding sub-paths each having a starting transit point that is the terminal transit point of the previous sub-path.

2. A method of chiropractic treatment according to claim 1, wherein the path has four sub-paths, the transit points of which are two transit points that are in horizontal alignment with one another and in horizontal alignment with the center of the circle and two transit points that are in vertical alignment with one another and in vertical alignment with the center of the circle.

3. A method of chiropractic treatment according to claim 1, further comprising placing a pillow beneath the shoulder of the person and another pillow beneath the buttocks of the person.
